(19) United States
(12) Patent Application Publication
(10) Pub. No.: US 2026/0249100 A1
(43) Pub. Date: Aug. 27, 2026

(54) METHOD FOR FINDING A TREATMENT PLAN AMONG RECOMMENDED PLANS FOR TREATING CANCER, USING AN INTERACTIVE FILTER (METHOD, TECHNICALLY FUNCTIONAL GUI)

(71) Applicant: FRAUNHOFER-GESELLSCHAFT ZUR FOERDERUNG DER ANGEWANDTEN FORSCHUNG E.V., Muenchen (DE)

(72) Inventors: Michael BORTZ, Kaiserslautern (DE); Karl-Heinz KUEFER, Weilerbach (DE); Philipp SUESS, Budenheim (DE); Katrin TEICHERT, Kaiserslautern (DE)

(21) Appl. No.: 18/694,325

(22) PCT Filed: Sep. 26, 2022
(86) PCT No.: PCT/IB2022/059067

§ 371 Date: May 11, 2026

(30) Foreign Application Priority Data
Sep. 25, 2021 (DE) 10 2021 124 813.2

(51) Int. Cl. A61N 5/10; G16H 20/40

(52) U.S. Cl. CPC A61N 5/1031; G16H 20/40; A61N 2005/1074

(57) ABSTRACT

The ability to improve the adjusting of technical parameters in therapy plans found by Pareto navigation constitutes a technical problem, along with facilitating the decision-making of the planner (also called user) and simplifying the mathematical calculation of the solution. Proposed is a way to find and establish a therapy plan among a plurality of plan proposals for cancer therapy, each plan proposal representing as a solution one point in a navigation space (N) containing a plurality of setting parameters and establishing in a machine space (M) a setting for radiation doses to be administered by a radiation therapy machine for cancer therapy. The navigation space (N) contains at least one Pareto front (200) behind or on which available treatment plans are located as possible solutions and among which a user navigates, whereby a first plurality of available solutions calculated in advance as plan proposals is provided in a memory and retrieved or read out from the memory for display. The retrieved or read-out plan proposals are shown on a display and used by the user for navigation, whereby a large number of Pareto-optimal solutions with physical values or evaluations of dose values can be found among the points. Moreover, such solutions are also shown that are almost Pareto-optimal, within a tolerance range of at most ±5% from the Pareto optimum. In this way, a large first set of points with the physical values or evaluations of dose values is created and then stored in the memory. From this, a second set of points is calculated, which defines clinical targets. A decision space (D) is provided and shown in which at least the clinical targets are plotted on target axes (76, 876, 776). This is without the possibility of navigating in the decision space (D) on the target axes of the clinical targets, whereas binary selections in the decision space are made in the manner of restrictors to specify which plan proposals are not used as therapy plans. This enables a desired therapy plan to be found and determined from the large number of plan proposals.

The present disclosure (and claims) relate to a method of multi-criteria optimization (MCO). Also affected is a GUI, either stand-alone or for application (use) in this procedure.

The invention relates to a method for optimizing a set state of a machine for tumor treatment (RT machine, radiation device, or therapeutic device). In particular, this involves a significant improvement of a system according to U.S. Pat. No. 7,391,026 B2. The control disclosed there of a planning process to determine the best possible plan for treating a patient suffering from a tumor disease was a quantum leap. It was a reversal of the previously proposed procedures and is known in the field for a special case of radiotherapy under IMRT. The procedure called “inverse therapy planning” was proposed by Bortfeld; see U.S. Pat. No. 7,391,026 B2, column 1, line 43 ﬀ. Over time, application and practical testing have opened up opportunities for achieving improvements in navigation, decision support, and the accuracy of the solution found as a therapy plan. Another matter of interest is how the mathematical calculation of the solution or solutions to be found has been simplified.

For purposes of this disclosure, a solution is a selected plan that is to be set or specified as a therapy plan. The user or planner (user for short) has a large number of plan proposals, all of which also represent solutions, but which still require optimization or, let's say, which open up opportunities for improvement.

Based on the aforementioned state of the art, the user can find a suitable plan using a design tool. The appropriate plan is far-reaching and contains setting parameters for making adjustments to (or on) a therapeutic device, which is later used to treat the tumor in a patient. Accordingly, it is clear that the process of setting this device and the preparation or determination for setting the device does not yet constitute therapeutic treatment or a medical treatment per se, but is a preliminary stage.

Technical setting parameters are found that allow radiotherapy to be performed later and possibly at a completely different location. This solution calls this radiotherapy a plan. IMRT is a special form of such radiotherapy. The latter term is the general treatment. The plan is also “a solution” selected from a large number of pre-calculated plans or solutions, all of which are suitable, whereas only one of them represents the best possible solution for the user. According to U.S. Pat. No. 7,391,026 B2, it is possible to select the best possible plan from the large number of plans (i.e., plan proposals) already available.

With WO 2012 069999 A1, the original plan (therapy plan or treatment plan) was only changed locally, not in its entirety. The expert would speak here of a local position-which can be described as small-being changed while “practically retaining the original plan as is.” If a new plan were to be used, the expert would not only change the localized critical ranges (or critical spots) but would also relocate them and not actually remove them by and large. An overly global change to the plan would therefore be avoided. In other words, changes to the “initial plan” that do not involve “critical spots” should be as few as possible.

WO 2013 093852 A1 provided the user with a more advanced, refined variant that deviated from the abstraction of U.S. Pat. No. 7,391,026 B2, which was available for navigation. The user or planner should be able to think in his/her practiced and conventional ways, and an important orientation in these practiced, conventional ways is offered by DVH arrays of curves, which provide the user/planner/doctor (user) with essential information for the quality of a “plan.” In the above-mentioned document, this DVH array of curves only had a representative function; it was (there in FIG. 6, bottom left) a sequence of a “plan” defined elsewhere.

The representation method of WO 2013 093852 A1 works with DVH arrays of curves, which are depicted in such a way that a user has it in (and at) hand for developing his/her plan proposals or to interactively find solutions from the stored range of solutions (corresponding to the plan proposals). The process visually displays a large number of pre-calculated solutions. This is done on a display device with a conventional screen. The large number of pre-calculated solutions for possible plans, which are stored in a database, are shown on the display device in such a way that the user is able to work with this range of solutions. A starting point is selected on one of the DVH curves. This starting point lies on the selected DVH curve, several of which are shown in the main diagram. This depiction, known as an “array of curves,” represents a solution of the database. The point is or will be selected as the starting point on a curve of this array of curves. The system assigns a straight axis to this point, which runs through the starting point. The straight axis intersects the 25 selected curve at the starting point or vice versa; the starting point defines both the position of the axis and the selected curve. A control area (as a navigation section or window) is highlighted around the starting point, which the user perceives as “visually highlighted.” The “effective visual highlighting” of this control area (section) allows the user to visualize the range of solutions stored in the database. The user is then given the opportunity to control how this range is depicted and to select this for display. The large number of solutions not currently shown (all solutions that are saved minus the one solution shown in the main diagram) can be visualized in this way. It is not yet shown, as only one of the many solutions is shown in the main diagram, but it is indicated by the control area, which is located around the starting point and highlighted visually.

WO 2005 035061 A2-Riker, Nomos undertook an IMRT optimization in a DVH environment, 131 and 117 there. The following brief description refers to the reference signs in FIG. 4. The optimization works with sliders and has an undo function (partial undo slider, 155 there). Partial undo slider 155 basically corresponds to an undo button that automatically adopts the last setting made by the user (reference sign taken from there). Furthermore, by default, slider 155 is located with its handle 157 in the rightmost position when the relevant isodose contour 162 is released, and corresponding scan window 160 displays the modified plan. If the user moves slider 157 all the way to the left, the algorithm completely reverses the change. When the user pushes slider 157 to the right again, the change is fully restored so that the user can see the effects of the change in real time, completely or incrementally, while sliding the slider handle back and forth. The allows the user to better understand the extent of the trade-offs. The user can select any intermediate point to see the plan configuration before the adjustment and the result of the adjustment. 10 The basics of how to navigate in a navigation space using the Pareto principles can be found in Monz et al 2008 Phys. Med. Biol. 53 985 (algorithmic foundation of interactive multi-criteria IMRT planning), authors Monz, Kufer, Bortfeld, and Thieke. Pareto navigation is an interactive multi-criteria optimization method based on the “selection” and “restriction” navigation mechanisms. The former selection allows the formulation of wishes, while the latter restriction makes it possible to exclude undesirable plans. They are implemented as optimization problems on the plan bundle-a set consisting of pre-calculated plans (also referred to as plan proposals here). They can be approximately reformulated so that their solution time is less than a fraction of a second. The user is given immediate feedback regarding his/her decisions. Pareto navigation makes it possible to manipulate the current plan and the set of plans under consideration in real time. Changes are triggered by simple mouse operations, such as on a navigation star or a group of operating aids shown on a display, in the example as sliders, each of which change one criterion or target value, thereby resulting in the navigation star or the other sliders being updated in real time (or, more accurately, the handle of the slider). In this way, dose depictions change during 25 planning with interactive feedback provided to the user. One of the plans found can or will then be selected to be preset or set on therapy devices. Only later, i.e. after the adjustment, will a patient be treated using this adjustment.

Since every Pareto-optimal plan in the plan bundle can be found with just a few navigation steps, an associated software navigator makes fast and targeted plan finding possible. The concept thereby allows multi-criteria optimization in real time and interactively with the user. Pareto navigation offers unprecedented real-time interaction, user-friendliness and plan diversity.

One technical problem of the invention disclosed here involves the ability to improve the adjusting of technical parameters in therapy plans found by Pareto navigation, in particular facilitating the decision-making of the planner (also called user) and/or simplifying the mathematical calculation of the solution.

Claim 1 or claim 22 provides a solution.

As a first aspect, the claimed invention opens up a new horizon for the user. The previous planner, who will continue to be called the user, has prepared two different types of plans and decision criteria for the final planner, who will be called the physician. Normal navigation in Pareto space enables navigation to goals that can originate from the dose distribution based on a DVH. These goals are physical values or assessments that correspond to the dose average of an organ, or more generally a volume of interest. In contrast to the planner, the physician will not tend to be interested in these goals alone but instead will primarily prefer clinical targets published in the specialist literature or from clinical studies, which, however, are not or not easily implemented in navigation and with the calculation of Pareto solutions.

Such clinical targets may be set by the physician who is finalizing the plan and wants criteria to be considered, for example that a certain percentage of an organ must receive a minimum dose of radiation. Another clinical target is that at least one of two existing organs needs to survive radiotherapy. And another clinical target is to start treatment as early as possible. Yet another of these clinical targets is that, for example, 99% of a tumor volume must receive a minimum radiation dose. Particularly logical links between objectives from the navigation space, such as the above-mentioned requirement to spare at least one of multiple organs of the same patient, cannot be included or implemented there using the tools of navigation.

When determining a therapy plan, the physician often speaks a different language than the user or planner who is focusing on physical parameters during navigation. Of course, the physician is also capable of dealing with these physical values, but will not primarily accept or query them as a goal. Among the goals preferred by the physician as clinical targets are the examples mentioned above, which are mathematically “non-convex” functions of the physical dose. However, it is difficult to arrive at an optimal solution to non-convex functions in a given time.

The solution lies in creating a decision-making space in which navigation in the usual sense is not possible, and where the physician's clinical targets are taken into account. The calculation of the above-mentioned clinical targets is based on the dose-volume constraints, i.e. the dose averages per organ (or, more generally, the volume of interest). From these dose values, such as the specification of a radiation dose >70 Gy for a (total) physical target, a clinical target is calculated, which evaluates the dose for, say, 99% of the specified volume in order to calculate a clinical target.

With this invention, a first large set of points, which need not only be Pareto, is obtained from the navigation, which is subsequently translated or converted to the clinical targets.

The more points there are, the higher the number of points in the decision area. This makes sense here and is actually necessary in order to have sufficient points for the clinical targets. Those that are almost Pareto can be sorted out. There is no 1:1 relationship between the points in the navigation space and the points in the decision space where the clinical targets are plotted. Even though the dose averages may be close to the clinical targets, direct transfer or mapping is not possible. And it is equally impossible to recombine based on the clinical targets, i.e. to calculate back to the physical evaluations of the dose averages.

The doctor does not find the most suitable therapy plan from the multitude of plan proposals using navigation, but rather using restrictors in the decision space to deselect plan proposals in order to arrive at the desired plan or target plan. The larger the first set of points translated or converted from the navigation space into the decision space is, the more accurate its selection can be. Navigation in the navigation space offers the possibility to do this, as the user employs convex combining to create enough solutions, which can be saved and converted into the decision space.

Interpolation is used during navigation to create new points from the points that have already been calculated previously. The conversion of all points for the decision space can eliminate those that do not have a Pareto property after the conversion. If you want to further increase the number of points used as a starting point, you can also use points that do not have a Pareto property. It is by no means guaranteed that Pareto points on the input side will also lead to Pareto points on the output side, i.e. the decision space.

The tolerance range mentioned in the claim that can form around the Pareto optimum is brought about by calculation inaccuracy, positional inaccuracy, model error, or numerical inaccuracy.

Target axes that specify a dose average for a volume of interest can also be plotted in the decision space (in general: a mathematical (convex) norm of the dose).

In particular, logical links between destinations cannot be calculated in a convex combination and are therefore not suitable for the navigation space. The following are examples of such goals: One of the salivary glands needs to survive the radiotherapy, or one of the two visual apparatuses needs to survive the radiotherapy, or one part of a double organ needs to survive the radiotherapy.

Even a passive goal is not suitable for the navigation space, such as the waiting time for the treatment.

The first set of points with the physical evaluations or values of doses can be greater than 1,000. This is regarded as the lower limit of a “large number of points.”

As mentioned above, no navigation occurs in the decision space, which is generally understood to mean selection and restriction. In the decision space, the physician is limited to restrictions but has sufficient choice due to the large number of plan proposals available as points. The physician can select or deselect entire technologies, and multiple target axes in the decision space can be grouped according to priority, for example.

Filtering in the decision space by deselecting unwanted plan proposals is carried out by restrictors able to deselect an entire contiguous area of plan proposals. To do this, a restrictor is set on a slider to deselect all points between itself and the end of the slider. Deselecting a slider causes the entire plan proposal to be deselected, along with the points on the other sliders.

A scoring function enables the physician to be presented with a solution as a plan proposal for each of several available technologies. This proposal is made automatically and, if the proposal is deselected by a restrictor, another proposal is automatically created from the available plan proposals in the decision space. The best possible proposal can be displayed, or the next proposal that is nearby based on the available solution set.

A “dose average” (as defined above) can be calculated differently depending on the organ. There are serial organs, such as the spinal cord, or parallel organs, such as the lungs, for which the dose averages are calculated differently.

According to another aspect of claim 22, a method is proposed for finding a “good” plan among many plan proposals (as possible solutions) for radiation-based cancer therapy, whereby each solution represents a point in a navigation space and forms a Pareto solution that includes- or defines-a plurality of setting parameters and defines in a machine space the settings for radiation doses that are to be delivered and the cancer therapy's course of radiation therapy at the therapy machine.

For the claimed solution, it is not intended that this set or specified therapy will actually be carried out. The solution claimed here is already complete when the settings are made or can be made on the therapy device (the therapy machine). A subsequent therapy or therapy that takes place along with the setting is not the subject of the claims. The invention described here is concerned with finding the best possible plan for a particular patient and, if necessary, specifying the setting parameters on the therapy machine.

The navigation space contains at least one Pareto front, behind which there are available treatment plans (plan proposals) which a user can navigate. All available solutions are stored in a memory and calculated in advance. They are retrieved or read in order to be shown from the memory, which can be a database. The retrieved or read solutions are shown on a display. The solutions available in this way are used by the user for navigation.

To navigate, the user can map the points of the plan proposals to target axes according to their destinations and can use sliders to do so.

A further, separately displayed decision space is also provided in which the navigable points from the navigation space are mapped to target axes without the possibility of navigating in the decision space, while the manner of a restrictor in the decision space allows binary selections to be made regarding which solutions are not used as therapy plans.

The decision space contains at least (one or more) such target axes that are not displayed in the navigation space for navigation. Target axes that are not suitable for the navigation space are those for which it cannot be mathematically guaranteed that the best possible solution is able to be calculated in a given time. These are outsourced to the decision-making area, which is where these target axes are located for use by the planner/user. Their use in the navigation space would make calculating predefined Pareto solutions more difficult—and at least lead to unexpected behavior for the user (during navigation)—as the sliders would not behave the same way as the planner/user moves them.

And yet this does not mean that no target axes are displayed in the decision space that are also displayed for navigation in the navigation space, i.e. the target axes actually displayed in the navigation space for navigation. Target axes that are provided in the navigation space only to circumvent the wording of the claim are to be considered the same as target axes not shown there.

With target axes, the criteria mapped on the target axes (including goals) can be taken into account in the restriction with which the user excludes such plans. In the decision space, this is to be seen as filtering—it is not navigation. The navigation itself or, more precisely, the Pareto navigation takes place in the navigation space, where quasi-constant changes to the plans are possible, an interpolation between neighboring points can be calculated, and the user can factor in the criteria that primarily influence the radiation (as goals) on target axes and use them for navigation. The concept of quasi-constant changes is almost continuous with regard to the screening or discretization of the plan proposals by Pareto navigation and the possible interpolation between adjacent points.

Examples that are not suitable for navigation include the following “coarser goals.” The time until a possible treatment on one or each of the multiple available therapy machines (time to treatment, or TTT), or a logical linking of goals from the navigation space. For example, one such link is the condition “at least one of the two salivary glands needs to survive radiotherapy, the left or right salivary gland, L-parotid or R-parotid.”

Quasi-constant navigation is possible for points in the navigation space whose navigation criteria can be applied to operating aids, such as to sliders. The plotted points from the memory enable the interpolation of intermediate values between neighboring points, which are only stored temporarily during navigation.

Target axes with criteria from the navigation space for which the above-mentioned quasi-constant navigation is possible can also be displayed in the decision space. This is not intended to contradict feature (d), as explained above.

Navigation in the navigation space and filtering in the decision space are made possible for the user by display on a visual device, such as a screen, tablet, or projection on a screen or canvas, whereby a continuously guided interaction between man and machine becomes—or is supposed to become—the basis for decision-making. Through the navigation, the user can interactively see the changes made to the criteria in the user's plans, including with consequences for the other criteria. Filtering in the decision space makes it “tangibly visible” to the patient which therapy proposals remain if the physician rejects or hides existing therapy proposals by making a binary selection, in particular by deselecting entire ranges on at least a few target axes, by filtering in effect.

A binary selection is preferably made in the decision space by specifying an entire range to be filtered out. The entire range is defined by specifying a restrictor. To the left or right of this restrictor, the entire range is deselected or filtered out up to the end of the operating aid displayed.

Both longitudinally extending operating aids, in particular the above-mentioned “sliders,” as well as rotating operating aids are suitable. With sliders, there is a start and an end to the operating section or the sliding section, along with an operating button for making the setting (as a handle) that can be displayed visually.

The control knob is preferably the restrictor (also called the “handle”). If it is moved from one end of the sliding section, its rest position, all solutions in the decision space between its position at which the handle is released or placed, or “set,” and the end of the sliding section are filtered out. This applies to both sides of the slider.

Preferably, different ranges can be specified on different target axes in the decision space and thus filtered out.

Preferably, target axes can be grouped in the decision space. Grouping or prioritizing the goals is a good way to improve the clarity and speed of finding the best possible therapy plan.

For example, as follows:
must-have goals,
important goals,
infeasible goals that cannot be arrived at by navigation.

A group of technologies can be mapped in the decision space, with each technology representing a therapy machine, such as a machine that works with protons or one that works with electrons or another that works with photons. Heavy ions can also be used as beam particles in yet another machine. In this group, each technology can be selected or deselected, which also corresponds to filtering.

Therapy plans of a deselected technology are no longer displayed in all sliders.

Variants within a technology can also be represented as a separate group, such as different irradiation configurations or other technical parameters of a photon treatment.

In the decision space—in addition to a graphical visualization of the planning proposals that were not selected—either the remaining planning proposals can be highlighted or the filtered-out planning proposals can be visually disqualified. For example, this can be done by employing a shade of gray, by otherwise changing the color, or by hatching or structural changes, preferably along the slider.

According to claim 34, points from two different navigation spaces can be mapped in a common decision space. This option makes it possible to compare fundamentally different therapies, such as chemotherapy and radiotherapy, which have fundamentally different navigation spaces but which can be evaluated according to risk and success, for instance (these would be two clinical targets in the decision space).

Another function is a scoring function that highlights certain plan proposals from among the remaining ones. This can be done by marking on the target axes, for example with a marker or a wedge, whereby the scoring function proposes only one plan for each technology if multiple technologies are available, in particular the best possible plan proposal from the scoring function's point of view. By changing the filtered plan proposals, either entire technologies can be deselected, or filtered out, or only portions of solutions still displayed may be deselected or filtered out on a slider. The latter then will not affect the entire technology, and the scoring function adds a new marking for the technology if the best possible planning just displayed is filtered out by setting the restrictor.

The numerical range of the solution set for a criterion can be displayed in the slider. This range data is updated when the user applies the filter to the solution (e.g. via a filter restrictor).

The range can be displayed in two ways:
(1) The numerical values for the minimum and maximum are displayed within the slider.
(2) A color shade is displayed within the slider indicating the available/filtered range of the criterion.

The optimal solution(s) can be displayed as said markers along the slider. The numerical value for all optimal solutions or only that of a global solution can be displayed.

For example, the scoring function yields a score weighted by priority or a lexicographical fulfillment of requirement levels (or both). The scoring function can be configured by setting priorities for decision criteria.

A structure—called a GUI—on a display, tablet, screen, or other “display device” for visibly identifying a functional control is described in claim 35. It is not a mere reproduction of information, but contains the functionality to make the changing of a plan “tangible” and visible to a planner/user by means of plan determination. Each point or slider shown stands for technical settings that represent dose, dose average (in general: as a mathematical (convex) norm of the dose), or at least an angle of incidence of a beam or temporary beam bundle in the machine space. These values are undoubtedly technical, even measured values or measurable values that are defined by higher-level specifications.

The technically usable graphical user interface (GUI) makes it possible to find a therapy plan among plan proposals for cancer therapy. Each solution as a plan proposal represents a point in a navigation space (N) and forms a Pareto solution containing a plurality of setting parameters (as outlined above) for determining, in a machine space, the settings for radiation doses to be delivered by a radiotherapy device (preferably a radiotherapy machine) for cancer therapy. At least one displayed navigation space is provided, which has at least one Pareto front, whereby a plurality of available plan proposals are held in a physical memory that have been calculated in advance and are retrieved or read out from the memory for display. The retrieved or read-out solutions are shown on a display and can be operated by the user using the GUI with the help of sliders for navigation. A further, separately displayed decision space is provided in which the navigable points from the navigation space are mapped to target axes, functionally without the possibility of navigating in the decision space, while the manner of a restrictor in the decision space allows binary selections to be exclusively made on the target axes in the decision space as a slider regarding which plan proposals are not usable or should not be usable as therapy plans. The decision space contains and displays at least those target axes for filtering that are not displayed in the navigation space (N) for navigation.

Illustrations of the GUI can be found in dependent claims 36 to 39.

Embodiment examples illustrate the invention without limiting it to these examples.

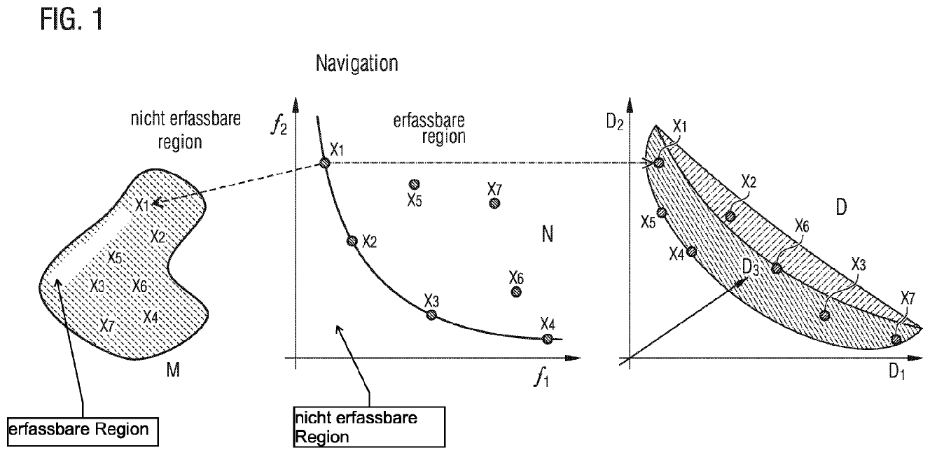

FIG. 1 illustrates three spaces with which a first example of the invention works. There may be another space N2 that is not shown, but its criteria can also be converted to decision space D. D is a decision space, preferably shown in red. N is the navigation space. M is the “machine space,” preferably shown in a shade of blue.

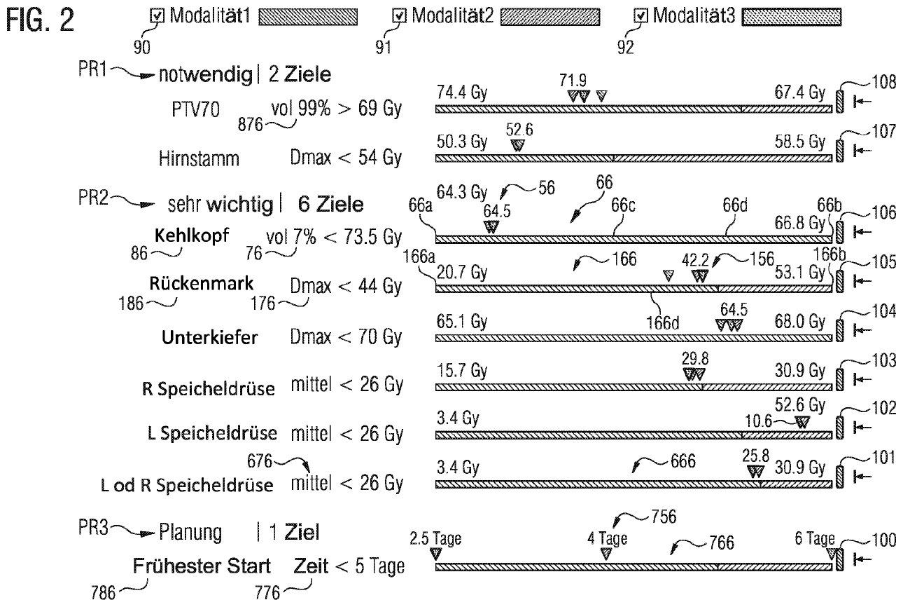

FIG. 2 shows an initial status of decision space D, along with its sliders.

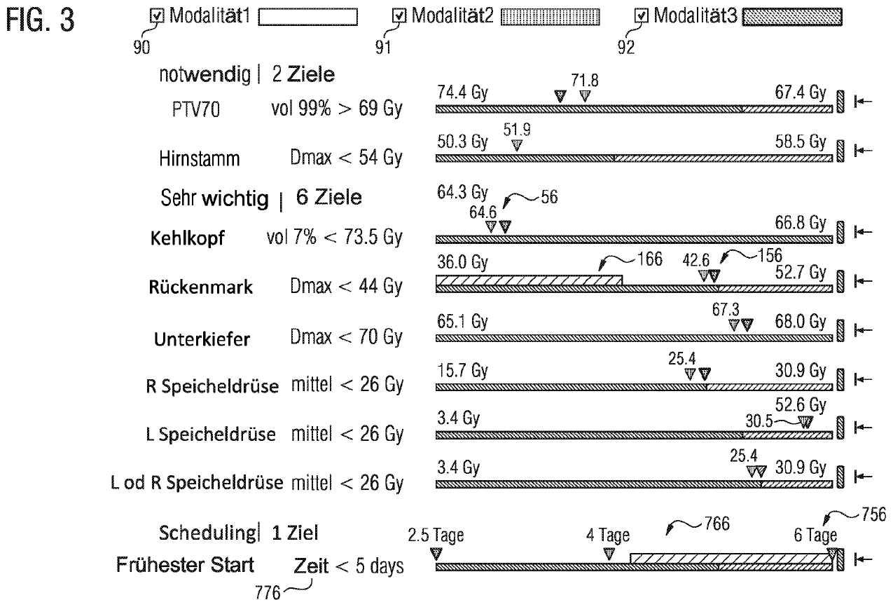

FIG. 3 illustrates a second status of decision space D, where a (whole) technology 90 is deselected.

An example not shown in FIG. 3 explains decision space D with several indented restrictors, where, for example, actuator 100 (a handle representing other handles) has been moved from rest position (e.g. right) to working position (e.g. left) and set there (or vice versa). Here in working position, the handle causes a restriction, i.e. excludes or filters out plan proposals.

FIG. 1 shows a (first) navigation space N in the center. To the left is machine space M (the therapy machines), and to the right is decision space D. This representation is an abstraction and shows navigation space N with its solutions x1 to x7, which are to be seen here as an example of all available solutions. These solutions are mapped into the decision space D with the same name x1 to x7 and, in the case of a conversion for setting a therapy machine (not shown), the technical setting parameters behind the point (i.e. represented by them) are transferred to the therapy machine.

Pareto function 200 can be seen in navigation space N, and the achievable therapy proposals can be seen as solution points at the top right. The organization of the points in a form suitable for navigation on sliders is not shown, but this is behind navigation space N. Examples of this are individual target variables on individual axes, with each target axis representing a slider. In this way, filtering can be carried out via the solution set if multiple sliders are linked. Changing a handle, such as a slider, also changes the positions of the other sliders or their handles during navigation. The latter control buttons can be seen as handles in the graphic area, and the general formulation is the elongated operating aid.

It works differently with filtering. Here, the plan proposals filtered out on one target axis are also removed on other target axes, so they no longer form an available set of Pareto solutions there. Filtering therefore covers all displayed values on target axes, even if only one of the target values on one of the target axes is filtered out. The handle as a slider is given a different function.

In the decision space, the point cloud, which is mapped there from navigation space N, is divided into two zones with different gray values. This represents the Pareto solutions (darker) and the non-Pareto solutions, which are not factored in for finding solutions. Among them is x2.

The score function can be understood on the basis of the points mapped in the decision space. If the user, preferably a physician, moves to the left on criterion D1 and filters out x3, the score function automatically creates a new marker on the corresponding slider that corresponds to x6. As perceived by the user, the marker on the slider “jumps” from x3 to x6 (without intermediate steps).

The arrows pointing away from point x1 symbolize the conversion to a different space.
(a) From navigation space N, point x1 with its setting is converted to machine space M, where it is also referred to as x1. It represents the setting, or a setting of a therapy machine. The same point x1 is converted to point x1 in decision space D, where it is available as a clinical target. The illustration with the points represents the illustration with the sliders used in the following figures.
(b) An example of a conversion from navigation space N to decision space D is described as follows.

We consider the case of compliance with at least one of two goals. The goals are convex (i.e. from navigation space N), whereby the overriding goal of fulfilling at least one of them can only be handled sensibly in decision space D.

One goal, called ZL, is to limit the average dose below 26 Gray (Gy) in the left salivary gland (L-parotid) and another goal ZR is the same limit in the right salivary gland (R-parotid). These goals are formulated mathematically as follows

$$ZL : \frac{1}{{n}_{S L}} {\sum }_{i ∈ {V}_{S L}} {d}_{i} \leq 26 Gy$$

$$and$$

$$ZR : \frac{1}{{n}_{S R}} {\sum }_{i ∈ {V}_{S R}} {d}_{i} \leq 26 Gy$$

Here, the dose is represented as a series of values dᵢ, as is generally the case, and index i represents a small volume fraction (“voxel”) in the patient's body—typically a small cube with an edge length of 3 mm. The indices of the voxels belonging to the left (right) salivary gland are shown with V_{SL} (V_{SR})—it is a list of voxel indices. The sum Σ_{i∈V}_{_{SL}} dᵢ thus represents the total sum of the dose in the left salivary gland (and the right one analogously). n_{SL} is the total number of voxels in the left salivary gland (n_{RL} analogously) by which the sum is divided to obtain the average dose. Both goals viewed separately are convex and can be handled in navigation space N.

The requirement to spare at least one salivary gland is now an overriding goal that we call ZS and is formulated as follows:

$$ZS : ZL ∨ ZR$$

Where “V” is the sign for “or” known from Boolean logic. Expressed as a formula, ZS can be equivalently described as follows:

$$ZS : χ ( \frac{1}{{n}_{S L}} {\sum }_{i ∈ {V}_{S L}} {d}_{i} \leq 26 Gy ) + χ ( \frac{1}{{n}_{S R}} {\sum }_{i ∈ {V}_{S R}} {d}_{i} \leq 26 Gy ) \geq 1$$

Where X(A) is a non-convex function that returns the value 1 for any event A if A is true and 0 if A is not true. The requirement in the above equation for ZS is therefore fulfilled if at least one of the two salivary gland goals is met.

In this way, the conversion can be made from navigation space N to decision space D, as shown in an example. The process is similar for other convex goals (i.e. from navigation space N).

FIG. 2 illustrates a decision space D with sliders, which is shown on a screen. The page is to be seen as a screen. Other forms of representation are a display or a touch-controlled tablet. A projector that projects a light signal onto a projection surface is also possible. It can be used from a second display, which can be operated by a finger with swipe and tap or by a mouse pointer.

On the display of FIG. 2, a target axis is shown in priority group PR3 as slider 766, which was not present in navigation space N. It was not used for navigation. Besides this slider as target axis 766, six additional sliders are also provided as target axes in priority group PR2 and are plotted parallel to one another. Two target axes are arranged above, which belong to priority group PR1. Three technologies are represented at the top, representing three real therapy machines according to the type of radiation they emit. In the example, it can be assumed that protons, electrons, and photons represent these three technologies 90, 91, and 92, preferably 90 in a dark shade of red, 91 in a shade of yellow, and 92 in a shade of blue. The differences are symbolized here with hatching and can also be designed as different colors as above.

Each of the technologies can be selected and deselected. The selected status is shown. As such, no technology is filtered out.

The order of the arrangement is not limited to this example. A different arrangement can also be provided, with the technologies on the right or at the bottom and the sliders able to be aligned vertically.

The structure of each slider is the same—it merely represents a different organ of cancer therapy.

Three of these sliders are explained below. On slider 66, which defines a target axis, the achievable radiation dose in Gray (Gy) is illustrated with a bar. The radiation dose ranges from 64.3 Gy to 66.8 Gy. These two values are given at end positions 66a and 66b of slider 66. Restrictor 106 is located on the right end position. According to the function, this restrictor can be moved to the left on the slider and thus on the target axis. Where it is set, it performs a filter function, whereby all values to the right of it and thus all associated solutions that are mapped on the target axes are filtered out.

The filter function can be displayed by darkening the filtered section in all of bar 66c. Slider 66 stands for the target volume “larynx,” which is labeled as 86. The corresponding dose specification is labeled as 76. The larynx should receive less than 73.5 Gy of radiation, in a volume fraction of 7%. Once narrow bar 66d parallel to the slider has met this requirement with all solutions, the slider is marked with a color indicating this next to bar 66c.

Other markings are also possible; as we mentioned above, it can be by a different hatching or different structure or different color.

Also shown is marker 56, which highlights a plan as the best possible plan from the scoring function for all three technologies and marks it with a triangle. The respective color or shade or structure can be adapted to respective technology 90, 91, and 92. In addition, the radiation dose value for the organ of the target axis can be indicated, in this case 64.6 Gy.

On the basis of slider 66, slider 166—located below it—will be explained. As a medical volume, it affects the spinal cord, which is marked at position 186. Position 176 specifies that the maximum dose may be <44 Gy. Bar 166d, which is parallel to bar 166c, has two colors, i.e. it is only in one color up to a value of 44 Gy and in the other color for values above 44 Gy. This is determined by specification 176, which specifies this value. Moving restrictor 105 to the transition between the two colors would result in the solutions that do not correspond to specification 176 being excluded, or filtered out.

The scoring function also shows the best possible value for each technology in this slider 166; this is section 156. The marking corresponds to the color, structure, or hatching of the marking of technology 90, 91, or 92.

Restrictor 105 can also be moved back above the value 44 Gy to exclude all higher dose values. It does not have to always be set to specification 176 but can allow more or less as well, i.e., exclude fewer or more of the solutions.

In the first group PR1, a tumor volume PTV70 is specified as the medical volume. According to the specification, this is to be exposed to a dose value of more than 69 Gy in 99% volume. Below this is the slider for the brain stem. Specified here is that the maximum dose is to be <44 Gy.

The two sliders described above, which fall under group PR1, are highly relevant. By setting restrictors 107 and 108 to positions in the slider and setting them there, the solutions that do not meet the specification can be excluded, which can be marked in color in the parallel bar, corresponding to 166d. Since all sliders are linked to each other via the solution set, the removal of solutions by a restrictor automatically removes the corresponding values on the other sliders as well. If a solution is excluded, it is dropped for all sliders.

A slider 766 still needs to be explained. It is not linked to a target axis that is or was intended for navigation in the navigation space. It indicates the earliest possible start of a treatment. The specification is that the time is to be <5 days, and property 786 indicates the parameter of the earliest possible start of the treatment. This target axis is not accessible to a quasi-constant change, so it is not used for navigation in navigation space N.

Another slider 666 also needs to be explained. It specifies the target axis where a logical linking of navigable targets from the navigation space takes place. The left or right salivary gland needs to survive the radiotherapy. This is set with the specification 676, according to which the mean value is to be <26 Gy. The two salivary glands are also mapped individually via the two target axes above and are available for filtering. The mean value of both the left and right salivary glands is to be <26 Gy

FIG. 3 illustrates the removal or filtering of technology 90. This is excluded by the user, resulting in the lost of the best range for the spinal cord in the decision space. This is indicated by a darker gray bar section in slider 166, which is no longer available. Slider 766, which is missing a right-hand section, reacts in the same way by removing the bar section containing the six days that already violate the specified criterion. By doing this, the user has tightened the decision criteria.

The moving in of multiple “handles” (adjusting elements) 100, 101, 105, 107, and 108 of the slider is conceivable. The user has tightened up many criteria here. Not only do entire areas to the right of the respective handle drop out of consideration, but possible solutions on the other side of the handle are also filtered out due to the coupling of the sliders via the existing solution set.

Of the two handles 107 and 108, the area on the right is filtered out, which in any case does not comply with the specification that at least 69 Gy must be given in 99% of the volume for the target PVT70 and that the maximum dose in the brain stem must not exceed 54 Gy.

Handles 107 and 108 are also provided on the sliders, where the modality as technology 91 suggests a dose value of 71.4 and 52.2, respectively, as the score function. This dose value remains after a number of other proposed solutions are deselected, represented by dark gray bars 866c and 966c. As already mentioned, it was not achieved by navigation but by deselecting a large number of proposed solutions, by setting restrictors 108, 107, 105 and 101, 100.

Clinical target 876 therefore can be met. A physical evaluation of an average dose value can be found in slider 966 with the specification that the brain stem must receive a dose value of <54 Gy. Target 76 is once again a clinical target, as less than 7% of the volume of the larynx may receive a dose <73.5 Gy. All values plotted on slider 66 meet this criterion.

The clinical targets are grouped together in FIGS. 2 and 3. Two objectives are assigned to the necessary objectives. Six goals are assigned to the important goals, and one clinical target is assigned to priority group PR3. It depends on the
user, such as the physician, whether the patient perceives this goal as important, so it has a kind of special status.

Not shown is the example in which a second navigation space N2 is present, which represents a fundamentally different kind of therapy, such as chemotherapy. The criteria of the navigation space specified there are converted to the clinical targets of the decision space using an alternative conversion, namely the same decision space D into which the physical values or evaluations of the volume of interest from navigation space N are also converted to the clinical targets of decision space D and displayed in order to compare these two fundamentally different therapies in decision space D.

1. Method for finding and establishing a therapy plan among a plurality of plan proposals for cancer therapy, each plan proposal representing as a solution one point in a navigation space (N) containing a plurality of setting parameters and establishing in a machine space (M) a setting for radiation doses to be delivered by a radiation therapy machine for cancer therapy;
(a) the navigation space (N) contains at least one Pareto front (200) behind or on which available treatment plans are located as possible solutions and among which a user navigates, whereby a first plurality of available solutions calculated in advance as plan proposals is provided in a memory and retrieved or read out from the memory for display;
(b) the retrieved or read-out plan proposals are shown on a display and used by the user for navigation, whereby a large number of Pareto-optimal solutions with physical values or evaluations of dose values can be found among the points. Moreover, such solutions are also shown that are almost Pareto-optimal, within a tolerance range of at most ±5% from the Pareto optimum.
(c) whereby a large first set of points with the physical values or evaluations of dose values is created and is stored in the memory. From this, a second set of points is calculated, which defines clinical targets;
(d) whereby a decision space (D) is provided and shown in which at least the clinical targets are plotted on target axes (76, 876, 776);
without the possibility of navigating in the decision space (D) on the target axes of the clinical targets, whereas binary selections in the decision space are made in the manner of restrictors to specify which plan proposals are not used as therapy plans, thereby allowing a desired therapy plan to be found and established from the plurality of plan proposals.

2. Method according to claim 1, whereby such target axes (166) are also plotted with their criteria (176) in decision space (D) that contain physical values of dose values, in particular for a volume of interest indicating an associated dose average as a mathematical (convex) norm of the dose.

3. Method according to claim 1, whereby there is a target axis (766) among the target axes plotted in decision space (D that, as a passive target, indicates a time period for one of a plurality of therapy machines at which treatment on this therapy machine is possible or before which treatment on this therapy machine is not possible.

4. Method according to claim 1 or 3, whereby there is a target axis (666) among the target axes plotted in the decision space (D) that represents a logical combination of goals from the navigation space (N), in particular a condition
at least one of two salivary glands must survive the found and established radiotherapy; or
at least one visual apparatus of two visual apparatuses must survive the found and established radiotherapy, whereby one visual apparatus consists of the optic nerve and the eye; or
at least one organ of duplicate organs, such as the lungs, must survive the found and established radiotherapy.

5. Method according to one of the preceding claims, whereby the tolerance range around the Pareto optimum is predetermined by calculation inaccuracy, positional inaccuracy, model error, or numerical inaccuracy.

6. Method according to one of the preceding claims, whereby on at least one of the target axes with the clinical targets in the decision space (D), an entire range is deselected contiguously, caused by setting a restrictor (100, 101) pertaining to this target axis.

7. Method according to claim 6, whereby a further range is deselected contiguously, caused by setting a further restrictor (100, 101) pertaining to a further target axis in the decision space (D), whereby preferably different ranges on different target axes in the decision space (D) are filtered out.

8. Method according to one of the preceding claims, whereby multiple target axes are grouped in the decision space (PR1, PR2, PR3).

9. Method according to claim 8, whereby the grouping consists of necessary goals, important goals, and infeasible goals that cannot be arrived at by navigation in the navigation space.

10. Method according to one of the preceding claims, whereby a group of different technologies (91, 92, 93) is mapped in the decision space (D).

11. Method according to claim 10, whereby a scoring function automatically sets markers (56, 156) for certain plan proposals, in particular only one, preferably the best possible plan proposal for one technology each.

12. Method according to claim 11, whereby the marker (56, 156) is set on a slider (66, 166) with a restrictor.

13. Method according to one of the preceding claims, whereby it is not possible to calculate a recombination of the goals represented in the decision space (D) back into the navigation space (N).

14. Method according to any one of the preceding claims, whereby the first set of points with the physical evaluations or values of dose values is greater than 1,000.

15. Method according to one of the preceding claims, whereby the calculated second set of points, which respectively plots clinical targets, is converted to the decision space (D), but only those points that are Pareto-optimal in the decision space (D) are displayed.

16. Method according to one of the preceding claims, whereby points from the first set of points with the physical values or evaluations of dose values are plotted on sliders (66, 166) in the decision space (D), whereby each slider has a restrictor (106, 105).

17. Method according to one of the preceding claims, whereby the first set of points with physical values or evaluations of dose values is enlarged by interpolations between the retrieved or read-out plan proposals during navigation by the user; this enlarged first set of points is stored in the memory, and the second set of points defining clinical targets is calculated therefrom.

18. Method according to any one of the preceding claims, whereby the calculation of the second set of points does not correspond 1:1 with the first set of points.

19. Method according to one of the preceding claims, whereby there are target values in the first set of points with physical values or evaluations of dose values that represent a lower dose limit, an upper dose limit, or an average dose value.

20. Method according to one of the preceding claims, whereby the first set of points can be calculated according to convex functions during navigation in the navigation space (N).

21. Method according to one of the preceding claims, whereby dose averages are contained in the first set of points with physical values or evaluations of dose values, whereby these are calculated differently depending on the organ—according to the maximum norm for serial organs and according to the arithmetic mean for parallel organs.

22. Method for finding a therapy plan among plan proposals for cancer therapy, whereby each plan proposal as a solution represents one point in a navigation space (N) containing a plurality of setting parameters and establishing in a machine space (M) the settings for radiation doses to be administered by a radiation therapy device for cancer therapy;
(a) the navigation space (N) contains at least one Pareto front (200) behind or on which treatment plans are located as possible solutions and among and with which a user navigates, whereby a plurality of available solutions calculated in advance as plan proposals is provided in a memory and retrieved or read out from the memory for display;
(b) the retrieved or read-out plan proposals are shown on a display and used by the user during navigation;
(c) with a displayed decision space in which the navigable points from the navigation space are mapped to target axes (66, 166, 766) without the possibility of navigating in the decision space (D), while the manner of a restrictor in the decision space allows binary selections to be made regarding which plan proposals are not used as therapy plans;
(d) whereby the decision space contains and displays at least such a target axe (766) that is not displayed in the navigation space (N) for navigation.

23. Method according to claim 22, whereby such target axes (66, 166) with their criteria (76, 176) are plotted in the navigation space for which a quasi-constant change is possible during navigation.

24. Method according to claim 22, whereby there is a target axis (766) among the target axes displayed in decision space (D that indicates a time period for one of the therapy machines at which treatment on this therapy machine is possible or before which treatment on this therapy machine is not possible.

25. Method according to claim 22 or 24, whereby there is a target axis (666) among the target axes displayed in the decision space (D) that represents a logical combination of goals from the navigation space (N), in particular a condition according to which at least one of the two salivary glands needs to survive the radiation therapy or one of the visual apparatuses needs to survive the radiation therapy.

26. Method according to claim 22 or 23, whereby target axes with criteria from the navigation space (N) on which quasi-constant navigation can take place are also displayed in the decision space (D) but only for the binary selection of plan proposals.

27. Method according to one of the preceding claim 22 et seq., whereby entire ranges of plan proposals are deselected on at least some target axes, caused by setting a restrictor (100, 101) pertaining to the respective target axis.

28. Method according to claim 27, whereby different ranges can be specified on different target axes in the decision space (D) and thus filtered out.

29. Method according to one of the preceding claims, whereby target axes are grouped in the decision space (D) (PR1, PR2, PR3).

30. Method according to claim 29, whereby the grouping consists of necessary goals, important goals, and infeasible goals that cannot be arrived at by navigation.

31. Method according to one of the preceding claim 22 et seq., whereby a group of technologies (91, 92, 93) is mapped in the decision space (D).

32. Method according to one of the preceding claim 31, whereby a scoring function automatically sets markers (56, 156) for certain plan proposals, in particular only one plan for one technology each.

33. Method according to claim 32, whereby the marker (56, 156) is set on a slider (66, 166).

34. Method according to any of the preceding claim 22 et seq., whereby another navigation space contains at least one additional Pareto front, behind which or on which treatment plans lie as possible solutions of an alternative therapy and among which and with which the user navigates, whereby these are also converted to the same decision space (D) and are compared there according to clinical targets.

35. Technically usable graphical user interface for finding a therapy plan among plan proposals for cancer therapy, whereby each solution as a plan proposal represents a point in a navigation space (N) and forms a Pareto solution containing a plurality of setting parameters for determining in a machine space (M) the settings for radiation doses to be administered by a radiation therapy device for cancer therapy;
(a) having a navigation space (N) that has at least one Pareto front (200), whereby a plurality of available plan proposals are held in a memory, have been calculated in advance, and are retrieved or read out from the memory for display, whereby the retrieved or read-out solutions are shown on a display and can be used by the user for navigation with the aid of sliders;
(c) whereby a further, separately displayed decision space (D) is provided in which the navigable points from the navigation space are mapped to target axes (66, 166, 266) without the possibility of navigating in the decision space (D), while the manner of a restrictor in the decision space allows binary selections to be made on the target axes in the decision space as a slider regarding which plan proposals are not usable or should not be usable as therapy plans;
(d) the decision space (D) contains and displays at least those target axes (766) for filtering that are not displayed in the navigation space (N) for navigation.

36. Graphical user interface according to claim 35, whereby target axes with criteria from the navigation space (N) on which quasi-constant navigation is possible are also displayed in the decision space (D), in particular as sliders (766, 666).

37. Graphical user interface according to one of the preceding claim 35 et seq., whereby target axes are shown grouped in the decision space (PR1, PR2, PR3).

38. Graphical user interface according to claim 37, whereby the grouping consists of necessary goals, important goals, and in-feasible goals that cannot be arrived at by navigation.

39. Graphical user interface according to one of the preceding claim 35 et seq., whereby a group of non-identical technologies (91, 92, 93) is mapped in the decision space (D) that correspond to different radiotherapy machines in the machine space (M), in particular according to the type of their beams.
